(19) United States
(12) Patent Application Publication
(10) Pub. No.: US 2025/0367852 A1
(43) Pub. Date: Dec. 4, 2025

(54) SEQUESTRATION, CAPTURE, AND IMPLEMENTATION OF CARBON-BASED MATERIALS IN ASSOCIATION WITH CEMENTITOUS MATERIALS TO FORM ADDITIVE MANUFACTURED STRUCTURES

(71) Applicant: ICON Technology, Inc., Austin, TX (US)

(72) Inventors: Sean Monkman, Austin, TX (US); Daniel Galvez Moreno, Austin, TX (US)

(21) Appl. No.: 18/677,660

(22) Filed: May 29, 2024

(51) Int. Cl. B28B 1/00; B33Y 10/00; B33Y 50/02; B33Y 70/10; C04B 14/02; C04B 40/00; C04B 111/00

(52) U.S. Cl. CPC B28B 1/001; B33Y 10/00; B33Y 50/02; B33Y 70/10; C04B 14/022; C04B 40/0032; C04B 2111/00181

(57) ABSTRACT

Various embodiments relate generally to additive manufacturing and construction techniques to form structures with embodiments including computer software and systems, and control systems, and, more specifically, to a computing and a mechanical platform configured to receive a material with which to form a structure of programmable dimensions and deposit the material including carbon-based materials and carbon-based captured elements in a cementitious material to form an additively constructed structure, such as a three-dimensional (“3D”) formed structure.

### FIELD

Various embodiments generally relate to additive manufacturing and construction techniques to form structures with embodiments including computer software and systems, and control systems, and, more specifically, to a computing and a mechanical platform configured to receive a material with which to form a structure of programmable dimensions and deposit the material including carbon-based materials and carbon-based captured elements in a cementitious material to form an additively constructed structure, such as a three-dimensional (“3D”) formed structure.

### BACKGROUND

Advances in robotics, computing hardware, and software have contributed to various improvements to provide materials for the construction of any type of structure such as a wall by extruding one or more materials as a “bead” or longitudinally formed material. In some cases, materials may be deposited as three-dimensional (“3D”) printed structures.

In some cases, typical construction techniques have been directed to employ one or more materials to form structures limited to dimensions that form single-story structures or buildings. Known mechanisms and processes for forming longitudinally constructed structures have been affected by various environment factors, such as wind, temperature, atmospheric conditions (e.g., humidity), or any force causing displacement of the placement of material. Hence, typical mechanisms and processes tend to produce structures less successfully and fail to address carbon-related elements that may offset gaseous emissions that may affect global climate.

Thus, a solution is needed to convey and deposit materials to form one or more structures of various additive structures to combine carbon-related material with cementitious material to form any structure of any vertical or horizontal dimensions without the limitations of conventional techniques.

### BRIEF DESCRIPTION OF THE DRAWINGS

Various embodiments or examples (“examples”) of the invention are disclosed in the following detailed description and the accompanying drawings:

FIG. 1 is a diagram depicting a system configured to deposit a material to form a structure to sequester, capture, or implement carbon-based materials according to some embodiments;

FIG. 2 depicts an implementation of base mixer unit of FIG. 1, according to at least one example;

FIG. 3 is a diagram depicting base mixer unit logic configured to modulate constituent materials to form an additively constructed structure, at least in one or more embodiments;

FIG. 4 illustrates an exemplary application architecture for control for a nozzle unit to deposit or extrude one or more carbon-infused materials to manufacture a structure, according to some examples;

FIG. 5 depicts an example flow with which to deposit material using a material deposition device implementing carbon-infused material, according to some examples;

FIG. 6 depicts an example of a structure configured to sequester material, according to some examples;

FIG. 7 depicts an example of an example of a structure formed to include a portion for analysis to determine a subset of metrics, according to some examples;

FIG. 8 depicts an example of a flow with which to sequester a material in an additively formed structure, according to some examples; and

FIG. 9 illustrates examples of various computing platforms configured to provide various functionalities to components of a computing platform configured to provide functionalities described herein.

### DETAILED DESCRIPTION

Various embodiments or examples may be implemented in numerous ways, including as a system, a process, an apparatus, a user interface, or a series of program instructions on a computer-readable medium such as a computer-readable storage medium or a computer network where the program instructions are sent over optical, electronic, or wireless communication links. In general, operations of disclosed processes may be performed in any arbitrary order unless otherwise provided in the claims.

A detailed description of one or more examples is provided below along with accompanying figures. The detailed description is provided in connection with examples and is not limited to any particular example. The scope is limited only by the claims and numerous alternatives, modifications, and equivalents thereof. Numerous specific details are set forth in the following description to provide a thorough understanding. These details are provided for the purpose of example, and the described techniques may be practiced according to the claims without some or all of these specific details. For clarity, technical material that is known in the technical fields related to the examples has not been described in detail to avoid unnecessarily obscuring the description or providing unnecessary details that may be already known to those of ordinary skill in the art.

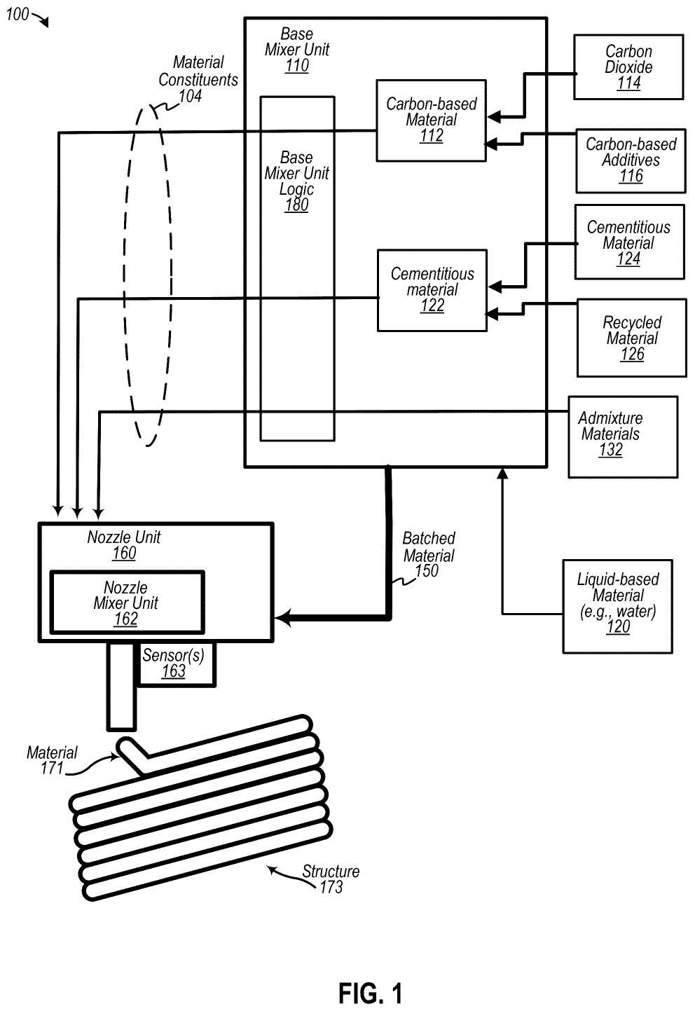

FIG. 1 is a diagram depicting a system configured to deposit a material to form a structure to sequester, capture, or implement carbon-based materials according to some embodiments. Diagram 100 depicts a system to apply or deposit material 171 to form a structure 173, such as a wall or any other constituent structure in the formation of an additive constructed three-dimensional (“3D”) structure, or otherwise described as a 3D printed cementitious-based structure (“3DPC”), which in some embodiments, may include carbon mineralization of carbon dioxide (“CO₂”).

As shown, diagram 100 depicts a base mixer unit 110 and a nozzle unit 160, including a nozzle mixer unit 162, any of which can be configured to generate a combination of carbon-related and cementitious material to form structure 173. One or more materials (in any amount or content) are depicted in diagram 100. As shown, any number of constituent materials may be combined to form structure 173. For example, gaseous, liquid, or any state of carbon dioxide 114 may be introduced to generate carbon-based materials 112. In some examples, carbon dioxide may be introduced as alternate CO₂ inputs through water soluble alkali carbonate minerals that may dissociate in water to form carbonate ions, such as Na2CO3, K2CO3, NaHCO3, KHCO3, CaHCO3, etc., as well as [NH4]2CO3 (i.e., as a soluble crystalline ammonium carbonate), among others. In various embodiments, the introduction of carbon or equivalent elements or molecules may enhance the reliability and strength of a 3D-printed cementitious based structure. Moreover, the introduction of carbon dioxide 114 may reduce the carbon footprint of embodied carbon in forming structure 173, and also may reduce operational carbon in the use and maintenance of structure 173 with subsequent usage after manufacture. In some examples, introduction of carbon dioxide 114 may also be added to nozzle unit 160. CO2 reaction with cement can modify the rheology and mixing in CO2 at nozzle mixer unit 162 prior to extrusion to, for example, beneficially reduce the flow. In other examples, soluble carbonates (e.g., water soluble alkali carbonate minerals as described above) may be added at nozzle unit 160. Sensor data could determine the quantity of CO2 to use. Sensors 163 may read the ambient temperature, the temperature of batched materials 150, quantitative aspects of the rheology (e.g., degrees or values representing slump, viscosity, yield stress, etc.) of batched material 150, a speed of the extrusion, and any other parameters. In at least one example, an amount of CO2 may be determined by the performance change that the amount of CO2 imparts. For example, the amount of CO2 may provide for a slump flow of material that changes from fluid (e.g., 22 to 30 cm) to self-supporting (e.g., 5 to 10 cm).

Carbon-based additives 116 may be combined to form carbon-based materials 112. For example, specific compounds, such as magnesium oxide, calcium oxide or equivalents thereof (e.g., CaCO₃), aluminum oxide, silicon dioxide, as well as any variants of fly ash may be combined to form carbon-based materials 112. In one example, fly ash may be formed subsequent to combustion of coal or other amounts of carbon. In various examples, carbon-based additives 116 may assist in reducing embodied and operational carbon in constructing a structure (e.g., residential housing, office buildings, military barracks, etc.).

Base mixer unit 110 may also be configured to receive cementitious materials 122 that may include any Portland cement, limestone, or any indigenous materials to form structure 173. Also, base mixer unit 110 may be configured to receive recycled materials 126, which may include any materials as well as carbon-reducing materials such as a proprietary building material produced by ICON Technology, Inc., of Austin, TX. Recycled materials 126 may include any size of aggregate crushed or reduced to a size as required.

Base mixer unit 110 may be configured to mix various amounts of materials to form material 171. For example, base mixer unit 110 may receive one or more admixtures 132. Admixtures may also be introduced prior to base mixer unit 110. In some cases, dry admixtures can be added to the dry mix materials (e.g., with the cement, SCMs, sand, Conex®, etc.). Dry admixtures introduced prior to base mixer unit 110 may also include soluble carbonates that may be activated upon mixing with, for example, water in base mixer unit 110. Further, any admixture may include any combination of a liquid viscosity modifying admixture (“VMA”), a shrinkage reducing admixture (“SRA”), a liquid-reducing admixture (e.g., a water-reducing admixture), a plasticizing admixture, an admixture to increase or decrease the air content of the mixture, a set accelerating admixture, and/or a retarding concrete admixture to set material 171 in various environmental conditions, and other types of admixtures, additives, binders, and the like to assist in deposition of discharged material 171. In some examples, material 171 may include at least a portion of a proprietary material produced by ICON Technology, Inc. of Austin, Texas, or any other equivalent material. Material 171 may be supplied to nozzle unit 160 via one or more material conduits (e.g., tubes, pipes, etc.), whereby nozzle unit 160 may be configured to mix or combine various components of a material adjacent nozzle unit 160, such as at a nozzle mixer unit 162, in some examples.

Base mixer unit 110 may be configured to receive any liquid 120, such as water or the like, to assist in mixing carbon-based material 112 and cementitious material 122 to form material 171.

Base mixer unit 110 includes base mixer logic 180 configured to receive sensor data (e.g., temperature, humidity, wind direction and forces, etc.), such as at sensor(s) 163. Sensors 163 may be configured to detect amounts of carbon-based material conveyed via nozzle unit 160 to form structure 173. Based on sensor data 163, base mixer unit 110 may be configured to modify the application of carbon-based material 112 as at least one of material constituents 104 as a function of detected carbon-based materials deposited. Base mixer unit 110 may also be configured to mix carbon-based materials 112 and cementitious materials 122 to provide batched material 150 at nozzle unit 160 or may control the mixing of carbon-based materials 112 and cementitious materials 122 (e.g., material constituents 104) at nozzle mixer unit 162.

In some examples, carbon-based additives 116 may include any material configured to facilitate re-carbonization whereby carbon dioxide may be absorbed (e.g., sequestered or captured) over time. In view of FIG. 1, greenhouse gases (“GHG”) may be reduced in terms of kg CO₂ per cubic yard relative to known cementitious materials used to form structures 173, such as a dwelling and the like. In one case, a reduced carbon footprint may be reduced to 280 kg CO₂ per cubic yard or less.

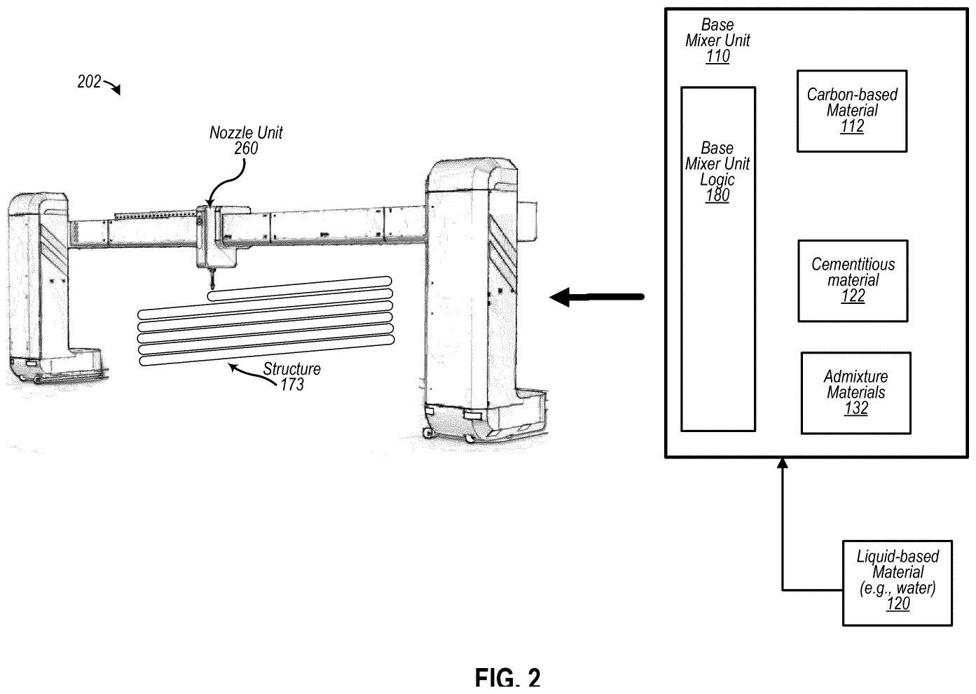

FIG. 2 depicts an implementation of base mixer unit of FIG. 1, according to at least one example. Material deposition unit 202 is shown to include a nozzle unit 260 to form structure 173 implementing base mixer unit 110, with constituent components described in FIG. 1. In some examples, material deposition unit 202 may be implemented as a Vulcan™ 3DPC device or a Phoenix™ 3DPC device manufactured or facilitated by ICON Technology, Inc. of Austin, Texas. In some examples, structure 173 may be formed consistent with standards, such as ICC-ES AC509 in accordance with guidance set forth by Applied Testing & Geosciences, LLC of Bridgeport, PA, USA.

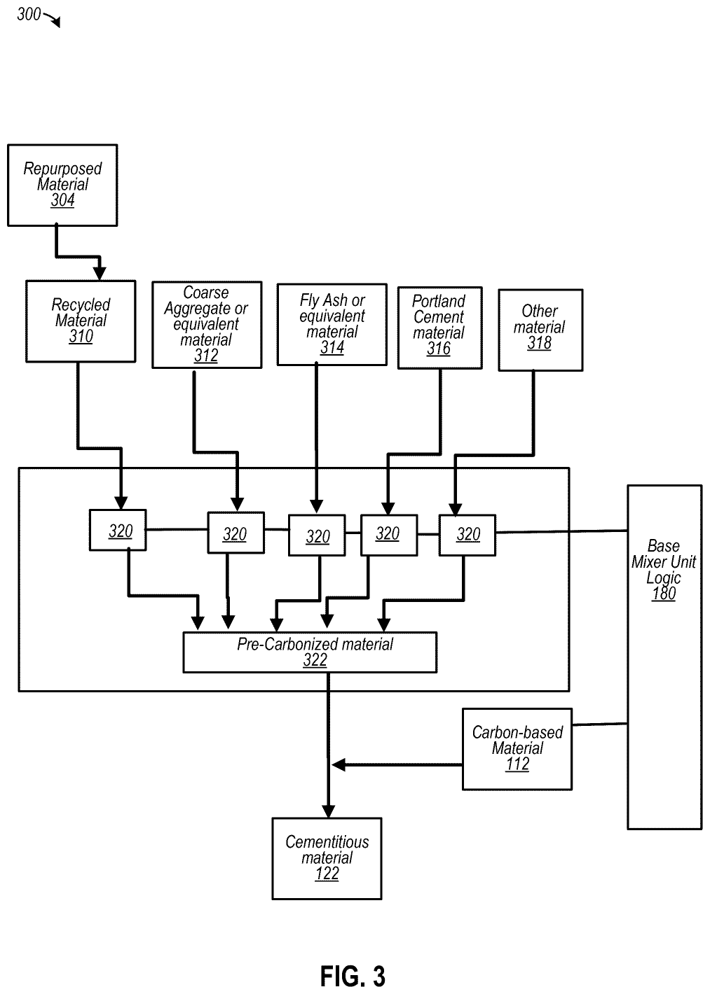

FIG. 3 is a diagram depicting base mixer unit logic configured to modulate constituent materials to form an additively constructed structure, at least in one or more embodiments. Diagram 300 depicts base mixer unit logic 180 configured to generate pre-carbonized material 322 via hardware, software, or the combination thereof by activating one or more selection devices 320 (e.g., switches, valves, etc.) to combine any constituent material in any proportion as a function, for example, of environment conditions (e.g., temperature, humidity, radiant energy (e.g., energized by sunlight), and the like).

Repurposed material 304 may include, for example, any form or base material derived or associated with Lavacrete™ that may be recycled by crushing and otherwise pulverizing said material for re-use and implementation as recycled material 310, which may also include any other material to form pre-carbonized material 322 to form an additively manufactured structure. In some examples, repurposed material 304 may also be carbonated as part of a recycling process. Further, repurposed material 304 based on recycled material 310 may be classified as a function of size fractions or dimensional sizes may be identified (e.g., comparable fineness to cement when used as a filler or binder, comparable fineness to a sand when used as a fine aggregate, comparable fineness to gravel if used as a coarse aggregate, and the like). For example, dimensional sizes and degrees of fineness may be determined by a grading curve produced through sieving. In one example, a fineness modulus may be calculated from the sieving results and used as a basis for comparison in view of other classifications of dimensional sizes.

Base mixer logic unit 180 may be further configured to introduce via selection devices 320 one or more of coarse aggregate (or equivalent material) 312, fly ash (or equivalent material) 314, Portland cement material 316, or another other material to form pre-carbonized material 322. Other material 318 may include formed one or more of limestone, clay, shells, silica sand, lime, gravel, mineral particulate, or any other material including any type of regolith. In some examples, material 318 may include other binder materials including, but not limited to, lag, silica fume, calcium sufflaminate cement, metakaolin, bottom ash, ladle slag, burnt oil shale, etc. Other materials may be included, such as reinforcing fibers (e.g., steel, nylon, polypropylene, PVA, glass, sisal, basalt, cellulose, etc.), and equivalents. Further, absorbent polymers (e.g., “super absorbent polymers”) may be added, and may include water-absorbing hydrophilic homopolymers or copolymers that may absorb and retain amounts of a liquid (e.g., extremely large amounts ”) relative to a mass of material, such as an absorbent polymer. Either carrying water (to potential reduce shrinkage of a printed material) or not carrying water (added at a nozzle and that may be used to reduce amounts of water in a printed material, which, in turn, rapidly modify a consistency and rheology, among other things).

In one example, recycled material 310 may include about 25% to about 30% by weight of the silica sand, about 30% to about 35% by weight of the combination of taconite powder and fine taconite aggregates, and about 20% to about 30% by weight of modifiable amounts of Portland cement and calcium carbonate. The remaining balance of about 5% to about 25% by weight may form a mixture of a liquid carbon-based nanoparticles solution with water, such as in a ratio of about 1:4. The recycled material 310 may also include additives or admixtures as well. A ratio of silica sand to taconite material is about 1:1 may be implemented in one example. Taconite material may include fine taconite aggregate and ground taconite powder at a ratio of about 1:1. A ratio of silica sand to fine taconite aggregate to taconite powder may be about 2:1:1, with varying ratios in any number of embodiments. Further, recycled material 310 may include any proportion of carbon-based materials (e.g., carbon dioxide, etc.). The above-described ranges are merely examples and may be implemented at any proportion.

Base mixer logic unit 180 may be configured to activate the introduction of carbon-based material 112 to instantiate carbon mineralization to form cementitious material 122.

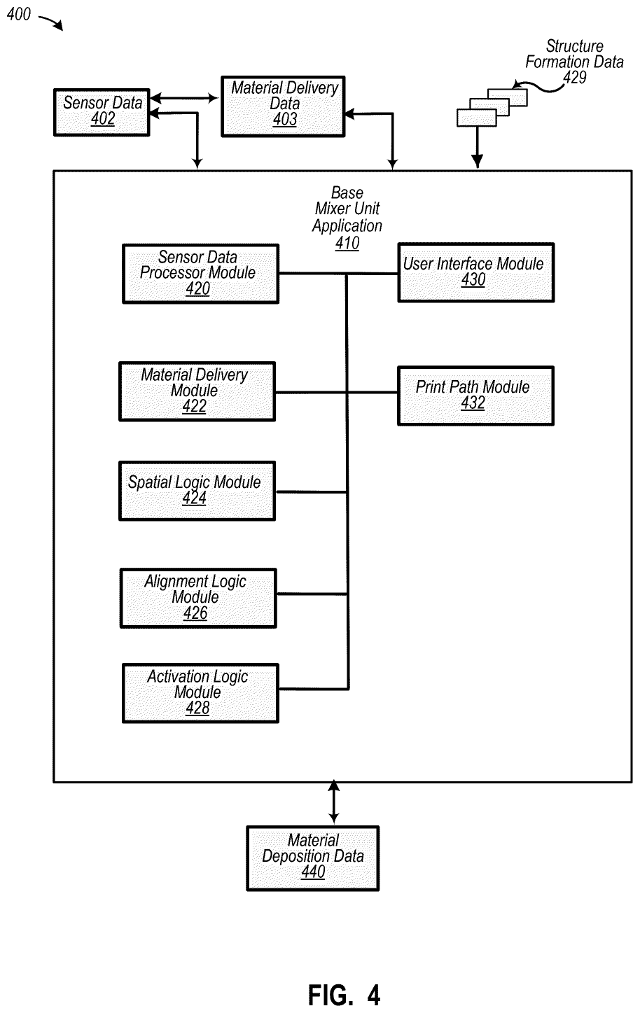

FIG. 4 illustrates an exemplary application architecture for control for a nozzle unit to deposit or extrude one or more carbon-infused materials to manufacture a structure, according to some examples. Diagram 400 is shown to include a base mixer unit application 410, including modules configured to provide functionalities based on sensor data 402 (e.g., carbon content), structure formation data 429 (e.g., data identifying a position or orientation of a print path), and material delivery data 403 configured to control delivery of a material through a nozzle unit, as well as any other data. Application 400 may be structured to generate material deposition 440 to control a nozzle by, for example, including 3D print commands or any other executable instruction to activate one or more actuators of a material disposition device (e.g., a nozzle). Structurally, in some examples, application 410 and the elements shown and described may be implemented as hardware, software, firmware, logic-specific circuitry, or as a combination thereof, without restriction or limitation to any particular implementation environment, 3D printing manufacturing process (or any other suitable manufacturing process), or configuration to form a structure like shelters, houses, buildings, roads, aircraft hangers, factory buildings, etc. Modules implemented in application 410 with substantially similar reference numbers may function to the other like-numbered elements shown and described herein including FIG. 1 and any other figure.

As shown in diagram 400, application 410 may include target spatial logic 912, which may include a sensor data processor module 420, a material delivery module 922, a spatial logic module 424, an alignment logic module 426, and an activation logic module 428, among others. Sensor data processor module 420 may be configured to receive a variety of subsets of sensor data 402 from any number of sensors to detect or compute a position and an orientation of one or more of a nozzle unit, a stabilization platform, and a material deposition device relative to a frame of reference. Material delivery module 422 may receive material delivery data at 403 and may be configured to monitor, implement, adjust, or otherwise control material flow as it is received into a nozzle unit or a material deposition device. Spatial logic module 424 may be configured to receive sensor data 402 and structure formation data 429, as well as any other data, to determine spatial dimensions of a print path associated with a surface (or other previously formed beads of material). Alignment logic module 424 may be configured to predict an alignment path over which the nozzle unit traverses to maintain the accuracy and precision of material deposition. Activation logic module 428 may be configured to interact with other modules to determine and control the activation of one or more actuators to control the position of a nozzle. In some cases, material deposition data 440 may be generated by activation logic module 428 or by any module in application 410. Note that each of the modules of application 410 may interact electronically with each other to correlate and/or combine functionalities to provide for material deposition. Further, any module may communicate internally or externally with other applications or other computing platforms via, for example, an application programming interface (“API”).

User interface module 430 may be configured to exchange data with any number of user interfaces for presenting activity data and for receiving instructions to view and modify functionalities of a material deposition device or any other portion of a manufacturing system. Print path module 432 may be configured to identify a print path and monitor the progress of material depositing to ensure conformance with manufacturing specifications and whether the predicted application of actuators to move a nozzle unit is within operating parameters. Non-conforming issues may be captured as data and transmitted via user interface module 430 to assist in troubleshooting.

Any of the described modules of FIG. 4 or any other processes described herein in relation to other figures may be implemented as software, hardware, firmware, circuitry, or a combination thereof firmware, logic-specific circuitry, or as a combination thereof, without restriction or limitation. Any of modules of FIG. 4 may be disposed, placed, distributed, or arranged in a material deposition device, such as a nozzle, or any module may be distributed at other portions of a system other than in a material deposition device.

If implemented as software, the described techniques may be implemented using various types of programming, development, scripting, or formatting languages, frameworks, syntax, applications, protocols, objects, or techniques, including, but not limited to, “G-Code,” Python™, ASP, ASP.net, .Net framework, Ruby, Ruby on Rails, C, Objective C, C++, C#, Adobe® Integrated Runtime™ (Adobe® AIR™), ActionScript™, Flex™, Lingo™, Java™, JSON, Javascript™, Ajax, Perl, COBOL, Fortran, ADA, XML, MXML, HTML, DHTML, XHTML, HTTP, XMPP, PHP, and others, including SQL™, SPARQL™, Turtle™, etc., as well as any proprietary application and software provided or developed by ICON Technology, Inc., or the like. The above-described techniques may be varied and are not limited to the embodiments, examples, or descriptions provided.

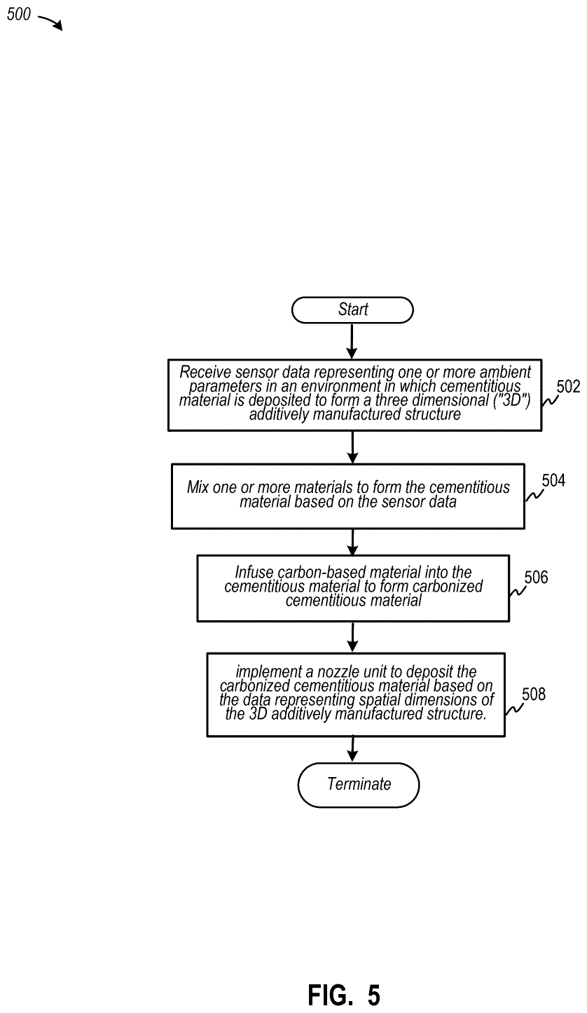

FIG. 5 depicts an example flow with which to deposit material using a material deposition device implementing carbon-infused material, according to some examples. At 502, flow 500 may be configured to receive sensor data representing one or more ambient parameters in an environment in which cementitious material is deposited to form a three-dimensional (“3D”) additively manufactured structure.

At 504, one or more materials may be mixed to form the cementitious material, for example, based on sensor data, whereby amounts of carbon dioxide and/or carbon association material may be infused into cementitious material as a function of one or more factors, including environmental or ambient factors (e.g., temperature, humidity, etc.).

At 506, carbon-based material may be infused into the cementitious material to form carbonized cementitious material. In one example, carbon dioxide or any other carbon-based material may be ‘injected’ into cementitious material used for 3D formation of additive structures.

At 508, a nozzle unit may be positioned or orientated to deposit carbonized cementitious material, for example, based on spatial dimensions of a 3D additively manufactured structure.

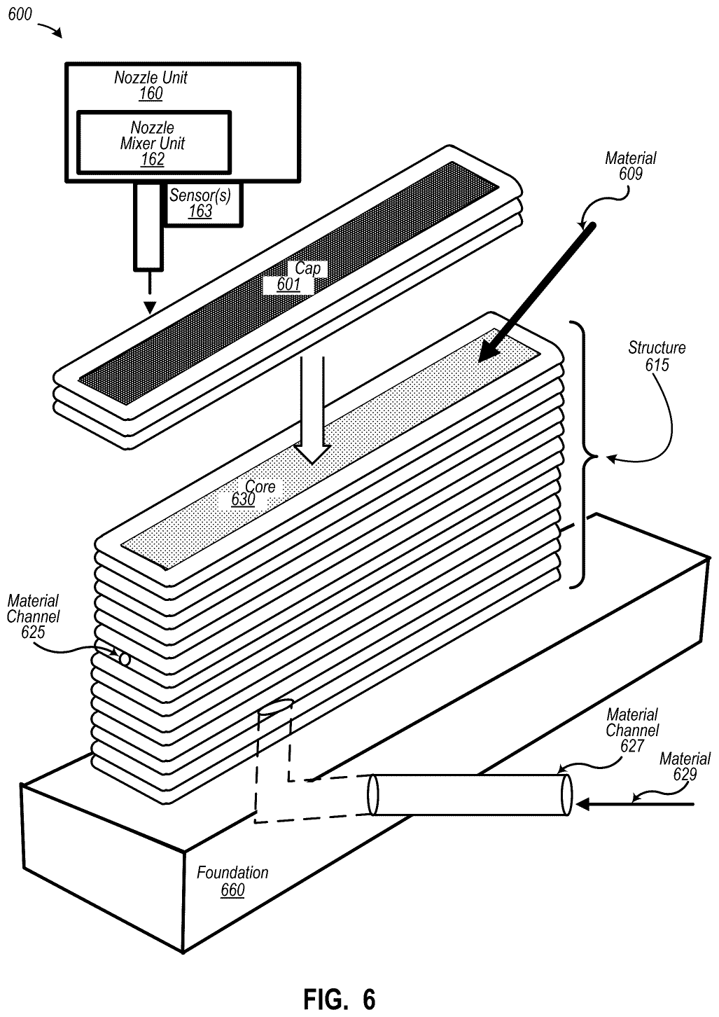

FIG. 6 depicts an example of a structure configured to sequester material, such as carbon dioxide, according to some examples. Diagram 600 shows a nozzle unit 160 including nozzle mixer unit 162 and sensors 163 of FIG. 1 configured to form structure 615 as a 3D additively manufactured structure configured to include a core 630 having a volume in which to dispose material 609, such as carbon dioxide. Structure 615, which may be formed as a wall, can be formed upon a foundation 660, such as a “slab” or any other foundational platform. In some examples, nozzle unit 160 may be further configured to form a cap 601, which may be a portion of structure 615 configured to “cap” or seal core 630 and its volume. Thus, core 630 may be sealed as an “airtight” structure or a reservoir with which to accept and maintain a material (e.g., a gaseous material, such as carbon dioxide or other compounds) within core 630.

Cap 601, structure 615, and foundation 660 can be formed to provide a volume in which a gaseous material may be introduced or “injected” for storage without leakage (or with substantially leakage) of the gaseous material external to a volume formed by cap 601, structure 615, and foundation 660.

In some examples, material 609 may be disposed as, for example, carbon dioxide in a solid, liquid, or gaseous state, as well as any combination thereof. For example, carbon dioxide or any other material 629 may be disposed in core 630 via material channel 627 via foundation 660 or may be disposed via material channel 625 in structure 625. In some examples, material 609 and 629 may include “CO₂ snow.” CO₂ snow injection may include liquid CO₂ converted into a solid or partially solid particulate including CO₂ snow particles with diameters of between, for example, 1 to 100 μm (or greater), via thermodynamic processes. The CO₂ “snow” particles may be derived from dry CO₂ ice having a temperature of approximately −78° C. (or a range +/−20° C.) and may propelled into core 630 via compressed air, as an example, or CO₂ gas. In some examples, material 609 and 629 may be provided by a tank, cylinders, or a mobile reservoir of CO₂ that may be used to inject material 609 and 629 via, for example, a “snow horn,” which is known. A flow meter or any other measuring device may be implemented to provide a sufficient amount of material 609 and 629 into core 630 regardless of whether the material is in a solid, liquid, or gaseous state, or any combination thereof.

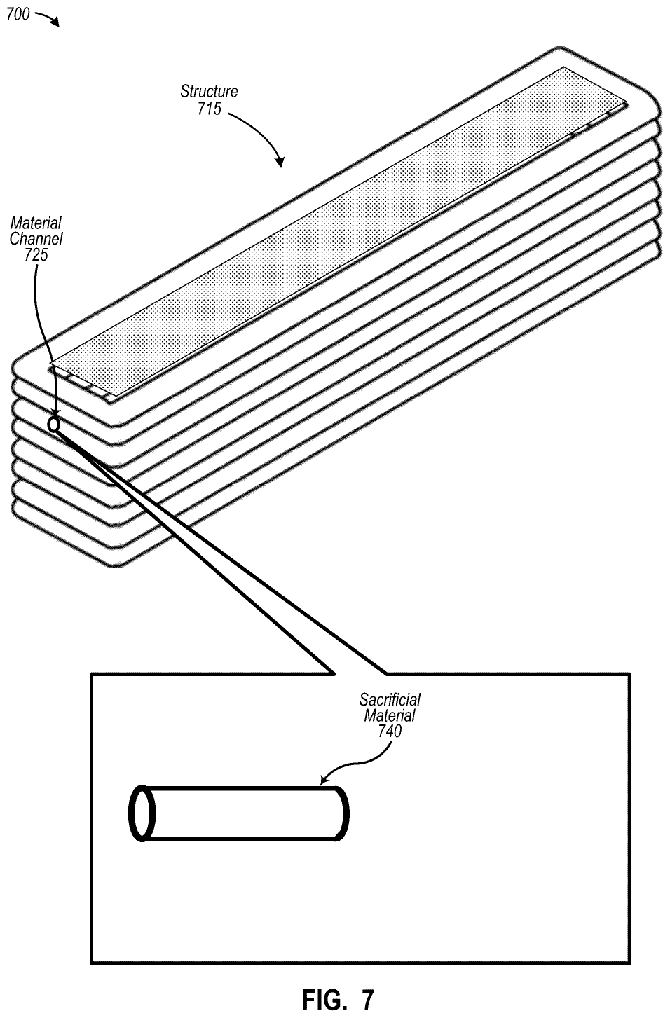

FIG. 7 depicts an example of an example of a structure formed to include a portion for analysis to determine a subset of metrics, according to some examples. Diagram 700 depicts a structure 715 including a material channel 725 (or any other orifice) that may be used as a sacrificial material 740. In some examples, sacrificial material 740 may be extracted from or at material channel (or any other portion of structure 715). In some examples, a cored section, such as sacrificial material 740, may be used or analyzed to determine degrees or levels of, for example, CO₂ mineralization and corresponding levels of carbon sequestration.

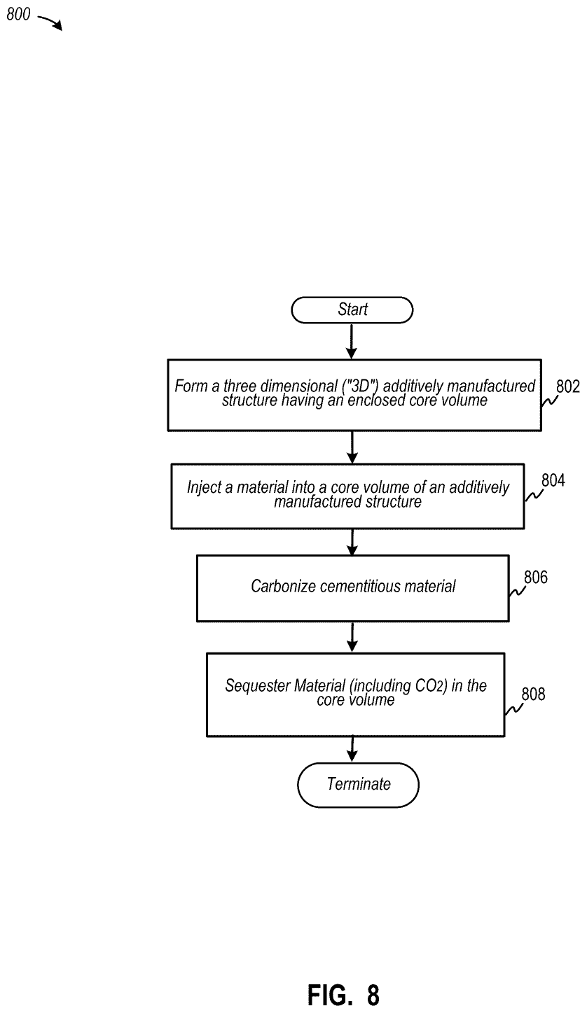

FIG. 8 depicts an example of a flow with which to sequester a material in an additively formed structure, according to some examples. At 802 of flow 800, a three dimensional additively manufactured structure is formed to include an enclosed core volume. At 804, a material may be injected into a core volume, whereby the material may include any state of CO₂ or any other element, molecule, or CO₂ or any other element, molecule, or substance. At 806, cementitious material may be carbonized with the introduction of CO₂ or any other element, molecule, or substance. At 808, material, such as carbon dioxide or equivalents thereof may be sequestered in a core volume.

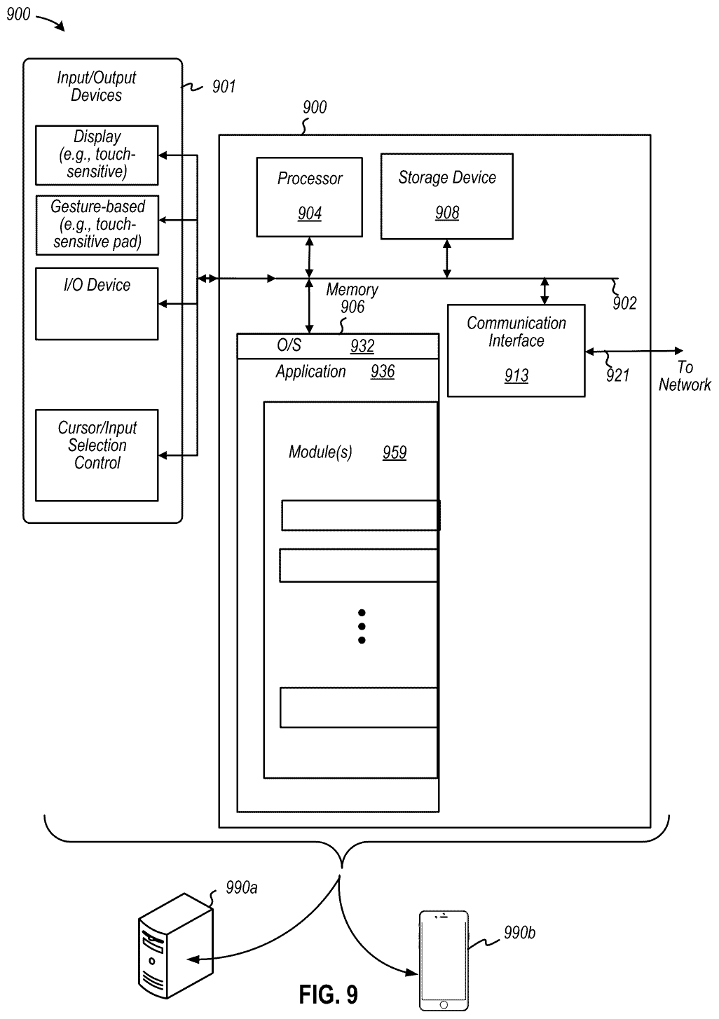

FIG. 9 illustrates examples of various computing platforms configured to provide various functionalities to components of a computing platform 900 configured to provide functionalities described herein. Computing platform 900 may be used to implement computer programs, applications, methods, processes, algorithms, or other software, as well as any hardware implementation thereof, to perform the above-described techniques.

In some cases, computing platform 900 or any portion (e.g., any structural or functional portion) can be disposed or located in any device, such as a computing device 990a, mobile computing device 990b, and/or a processing circuit in association with initiating any of the functionalities described herein, via user interfaces and user interface elements, according to various examples.

Computing platform 900 includes a bus 902 or other communication mechanism for communicating information, which interconnects subsystems and devices, such as processor 904, system memory 906 (e.g., RAM, etc.), storage device 908 (e.g., ROM, etc.), an in-memory cache (which may be implemented in RAM 906 or other portions of computing platform), a communication interface 913 (e.g., an Ethernet or wireless controller, a Bluetooth controller, NFC logic, etc.) to facilitate communications via a port on communication link 921 to communicate, for example, with a computing device, including mobile computing and/or communication devices with processors, including database devices (e.g., storage devices configured to store relational data, structured data, unstructured data, and graph data or atomized datasets, including, but not limited to triple stores, etc.). Processor 904 can be implemented as one or more graphics processing units (“GPUs”), as one or more central processing units (“CPUs”), such as those manufactured by Intel® Corporation, or as one or more virtual processors, as well as any combination of CPUs and virtual processors. Or, a processor may include a Tensor Processing Unit (“TPU”), or equivalent. Computing platform 900 exchanges data representing inputs and outputs via input-and-output devices 901, including, but not limited to, keyboards, mice, audio inputs (e.g., speech-to-text driven devices), user interfaces, displays, monitors, cursors, touch-sensitive displays, touch-sensitive inputs and outputs (e.g., touch pads), LCD or LED displays, and other I/O-related devices.

Note that in some examples, input-and-output devices 901 may be implemented as, or otherwise substituted with, a user interface in a computing device associated with, for example, a user account identifier in accordance with the various examples described herein.

According to some examples, computing platform 900 performs specific operations by processor 904 executing one or more sequences of one or more instructions stored in system memory 906, and computing platform 900 can be implemented in a client-server arrangement, peer-to-peer arrangement, or as any mobile computing device, including smartphones and the like. Such instructions or data may be read into system memory 906 from another computer-readable medium, such as storage device 908. In some examples, hard-wired circuitry may be used in place of or in combination with software instructions for implementation. Instructions may be embedded in software or firmware. The term “computer-readable medium” refers to any tangible medium that participates in providing instructions to processor 904 for execution. Such a medium may take many forms, including but not limited to, non-volatile media and volatile media. Non-volatile media includes, for example, optical or magnetic disks and the like. Volatile media includes dynamic memory, such as system memory 906.

Known forms of computer-readable media include, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can access data. Instructions may further be transmitted or received using a transmission medium. The term “transmission medium” may include any tangible or intangible medium that is capable of storing, encoding, or carrying instructions for execution by the machine, and includes digital or analog communications signals or other intangible medium to facilitate communication of such instructions. Transmission media includes coaxial cables, copper wire, and fiber optics, including wires that comprise bus 902 for transmitting a computer data signal.

In some examples, execution of the sequences of instructions may be performed by computing platform 900. According to some examples, computing platform 1500 can be coupled by communication link 921 (e.g., a wired network, such as LAN, PSTN, or any wireless network, including WiFi of various standards and protocols, Bluetooth®, NFC, Zig-Bee, etc.) to any other processor to perform the sequence of instructions in coordination with (or asynchronous to) one another. Computing platform 900 may transmit and receive messages, data, and instructions, including program code (e.g., application code) through communication link 921 and communication interface 913. Received program code may be executed by processor 904 as it is received, and/or stored in memory 906 or other non-volatile storage for later execution.

In the example shown, system memory 906 can include various modules that include executable instructions to implement the functionalities described herein. System memory 906 may include an operating system (“O/S”) 932, as well as an application 936 and/or logic module(s) 959. In the example shown in FIG. 9, system memory 906 may include any number of modules 959, any of which, or one or more portions of which, can be configured to facilitate any one or more components of a computing system (e.g., a client computing system, a server computing system, etc.) by implementing one or more functions described herein.

The structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. These can be varied and are not limited to the examples or descriptions provided.

In some embodiments, modules 959 of FIG. 9, or one or more of their components, or any process or device described herein, can be in communication (e.g., wired or wirelessly) with a mobile device, such as a mobile phone or computing device, or can be disposed therein.

In some cases, a mobile device, or any networked computing device (not shown) in communication with one or more modules 959 or one or more of its/their components (or any process or device described herein), can provide at least some of the structures and/or functions of any of the features described herein. As depicted in the above-described figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in any of the figures can represent one or more algorithms. Or, at least one of the elements can represent a portion of logic including a portion of hardware configured to provide constituent structures and/or functionalities.

For example, modules 959 or one or more of its/their components, or any process or device described herein, can be implemented in one or more computing devices (i.e., any mobile computing device, such as a wearable device, such as a hat or headband, or mobile phone, whether worn or carried) that include one or more processors configured to execute one or more algorithms in memory. Thus, at least some of the elements in the above-described figures can represent one or more algorithms. Or, at least one of the elements can represent a portion of logic, including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, modules 959 or one or more of its/their components, or any process or device described herein, can be implemented in one or more computing devices that include one or more circuits. Thus, at least one of the elements in the above-described figures can represent one or more components of hardware. Or, at least one of the elements can represent a portion of logic, including a portion of a circuit configured to provide constituent structures and/or functionalities.

According to some embodiments, the term “circuit” can refer, for example, to any system including several components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

1. A method comprising:
receiving sensor data representing one or more ambient parameters in an environment in which cementitious material is deposited to form a three-dimensional (“3D”) additively manufactured structure;
mixing one or more materials to form the cementitious material based on the sensor data;
infusing carbon-based material into the cementitious material to form carbonized cementitious material; and
implementing a nozzle unit to deposit the carbonized cementitious material based on the data representing spatial dimensions of the 3D additively manufactured structure.

2. The method of claim 1 wherein infusing the carbon-based materials comprises:
injecting carbon dioxide into the cementitious material.

3. The method of claim 1 wherein infusing the carbon-based materials comprises:
introducing carbon-reactive elements into the cementitious material to promote carbon mineralization.

4. The method of claim 1 wherein infusing the carbon-based materials comprises:
modifying amounts of the carbon-based material into the cementitious material as a function of the sensor data.

5. The method of claim 1 wherein receiving the sensor data representing one or more ambient parameters comprises:
receiving sensor data representing temperature, humidity, and displacement vectors indicative of wind; and
adjusting formation of the carbonized cementitious material based on the sensor data.

6. The method of claim 1 wherein mixing the one or more materials to form the cementitious material based on the sensor data comprises:
combining the cementitious material with one or more admixtures.

7. The method of claim 1 wherein mixing the one or more materials to form the cementitious material based on the sensor data comprises:
combining the cementitious material at or adjacent to the nozzle unit.

8. The method of claim 1 wherein mixing the one or more materials to form the cementitious material based on the sensor data comprises:
combining the cementitious material at or adjacent to a base mixer unit.

9. The method of claim 1 wherein implementing the nozzle unit to deposit the carbonized cementitious material based on the data representing spatial dimensions of the 3D additively manufactured structure comprises:
receiving a data file including data identifying a print path over which the nozzle unit deposits the material.

10. A system comprising:
a memory including executable instructions; and
a processor, responsive to executing the instructions, is configured to:
receive sensor data representing one or more ambient parameters in an environment in which cementitious material is deposited to form a three dimensional (“3D”) additively manufactured structure;
mix one or more materials to form the cementitious material based on the sensor data;
infuse carbon-based material into the cementitious material to form carbonized cementitious material; and
implement a nozzle unit to deposit the carbonized cementitious material based on the data representing spatial dimensions of the 3D additively manufactured structure.

11. The system of claim 10 wherein the processor configured to infuse the carbon-based materials is further configured to:
inject carbon dioxide into the cementitious material.

12. The system of claim 10 wherein the processor configured to infuse the carbon-based materials is further configured to:
introduce carbon-reactive elements into the cementitious material to promote carbon mineralization.

13. The system of claim 10 wherein the processor configured to infuse the carbon-based materials is further configured to:
modify amounts of the carbon-based material into the cementitious material as a function of the sensor data.

14. The system of claim 10 wherein the processor configured to infuse the carbon-based materials is further configured to:
receive sensor data representing temperature, humidity, and displacement vectors indicative of wind; and
adjust formation of the carbonized cementitious material based on the sensor data.

15. The method of claim 1 wherein the processor configured to mix the one or more materials to form the cementitious material based on the sensor data is further configured to:
combine the cementitious material with one or more admixtures.

16. A non-transitory computer readable medium having one or more computer program instructions configured to perform a method, the method comprising:
receiving sensor data representing one or more ambient parameters in an environment in which cementitious material is deposited to form a three dimensional (“3D”) additively manufactured structure;
mixing one or more materials to form the cementitious material based on the sensor data;
infusing carbon-based material into the cementitious material to form carbonized cementitious material; and
implementing a nozzle unit to deposit the carbonized cementitious material based on the data representing spatial dimensions of the 3D additively manufactured structure.

17. The method of claim 16 wherein infusing the carbon-based materials comprises:
injecting carbon dioxide into the cementitious material.

18. The method of claim 16 wherein infusing the carbon-based materials comprises:
introducing carbon-reactive elements into the cementitious material to promote carbon mineralization.

19. The method of claim 16 wherein infusing the carbon-based materials comprises:
modifying amounts of the carbon-based material into the cementitious material as a function of the sensor data.

20. The method of claim 16 wherein receiving the sensor data representing one or more ambient parameters comprises:
receiving sensor data representing temperature, humidity, and displacement vectors indicative of wind; and
adjusting formation of the carbonized cementitious material based on the sensor data.
